(19) United States
(12) Patent Application Publication
(10) Pub. No.: US 2026/0198543 A1
(43) Pub. Date: Jul. 16, 2026

(54) BIOAVAILABILE VITAMIN B12 IN SPIRULINA PRODUCTS

(71) Applicant: VAXA TECHNOLOGIES LTD, Rosh Pinna (IL)

(72) Inventors: Ohad BASHAN, Sde Varburg (IL); Isaac BERZIN, Jerusalem (IL)

(73) Assignee: VAXA TECHNOLOGIES LTD, Rosh Pinna (IL)

(21) Appl. No.: 19/550,725

(22) Filed: Feb. 26, 2026

Related U.S. Application Data
(63) Continuation-in-part of application No. 18/838,967, filed on Jan. 25, 2024.
(60) Provisional application No. 63/441,314, filed on Jan. 26, 2023.
(60) Provisional application No. 63/445,735, filed on Feb. 15, 2023.

(51) Int. Cl. A23L 33/135; A21D 2/26; A23J 3/20; A23J 3/22; A23L 33/15; C12N 1/20; C12P 19/42; C12R 1/01

(52) U.S. Cl. CPC A23L 33/135; A21D 2/267; A23J 3/20; A23J 3/227; A23L 33/15; C12N 1/20; C12P 19/42; C12R 2001/01

(57) ABSTRACT

Spirulina products, biomass, extracts, various food products, nutritional supplements, and methods of preparing and using the spirulina products are provided—in which bioavailable B12 outweighs pseudo B12. The spirulina products are prepared from Arthrospira spp. biomass that is cultivated under controlled, ultra-high-density conditions with strong UV illumination and strong continuous mixing. The spirulina products may be made of the algal biomass and/or wet/dry extracts thereof, and are characterized by high levels of unopposed B12, providing more bioavailable B12 than pseudo-B12. Spirulina products may have more bioavailable B12 than pseudo-B12 (e.g., at least two or three times more bioavailable B12 than pseudo-B12, and up to thousands of times more), providing a large portion of the required daily B12 intake in a broad range of food products.

### CROSS-REFERENCE TO RELATED APPLICATIONS

This application is continuation in part (CIP) of U.S. patent application Ser. No. 18/838,967 filed on Aug. 15, 2024, which is a National Phase Application of PCT International Application No. PCT/IL2024/050101, International Filing Date Jan. 25, 2024, claiming the benefit of U.S. Provisional Application No. 63/441,314, filed Jan. 26, 2023, and U.S. Provisional Application No. 63/445,735, filed Feb. 15, 2023, each of which is hereby incorporated by reference in its entirety.

### BACKGROUND OF THE INVENTION

### 1. Technical Field

The present invention relates to the field of artificially grown and extracted spirulina products and, more particularly, to providing spirulina products with bioavailable B12.

### 2. Discussion of Related Art

The physiologically active, bioavailable forms of vitamin B12, termed cobalamins, have a 5,6-dimethylbenzimidazole (DMBI) as the lower axial ligand coordinated to the central cobalt ion, such as in methylcobalamin and 5′-deoxyadenosylcobalamin. Physiologically bioavailable B12 is also termed bioactive or unopposed B12.

In contrast, biologically inactive pseudo forms of B12, termed cobamides have a different base (e.g., adenine) substituting for DMBI and are not bioavailable. It is noted that cyanocobalamin is a synthetic cobalamin that includes the DMBI base, but is not active due to its cyano group, which may be replaced physiologically with a methyl group or a 5′-deoxyadenosyl group to form the two active forms. Pseudo-vitamin B12 typically refers to compounds that are corrinoids with a structure similar to vitamin B12 but without vitamin activity, and is therefore metabolically unavailable.

Commercial spirulina algal extracts are typically made of cyanobacteria grown in open ponds and/or in other solar-based cultivation systems, and include the non-active, physiologically unavailable pseudo-B12, which opposes and reduces its bioavailability. For example, van der Oever and Mayer 2022 (Biologically active or just “pseudo”-vitamin B12 as predominant form in algae-based nutritional supplements?, Journal of Food Composition and Analysis, 10, 104464), incorporated herein in their in its entirety, teaches a simple and fast ultra-high performance liquid chromatography method with UV detection to measure algae-based nutritional supplements contain both physiologically active vitamin B12 (e.g., cobalamin in various forms, such as methyl-, cyano-, hydroxo- and adenosyl-cobalamin) and its non-active pseudo-form. The measurements show that in Spirulina-labeled nutritional supplements, pseudo-vitamin B12 prevailed at a wide range of concentration over physiologically active vitamin B12.

Accordingly, extracts in which pseudo-B12 forms dominate are actually anti-nutrients, obstructing or preventing absorption of physiologically active forms of B12 from other sources.

Spirulina algae (Arthrospira Spirulina platensis) cultivated in geothermally powered photobioreactors were proposed as a potentially resource efficient, zero-carbon, and nutritious alternative to conventional beef meat, e.g., in Tzachor et al. 2022 (Environmental Impacts of Large-Scale Spirulina (Arthrospira platensis) Production in Hellisheidi Geothermal Park Iceland: Life Cycle Assessment, Marine Biotechnology 24:991-1001), incorporated herein in their in its entirety. Furthermore, Tzachor et al. 2022 suggest direct decarbonization of food systems and diets by incorporating Spirulina from the geothermal park in meals as a beef meat replacement and indirect decarbonization of food systems by means of issuing, selling, and purchasing carbon credits between enterprises.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides spirulina products made of a water-based extracts of Arthrospira spp., cultivated under artificial conditions that include intense illumination and mixing—to yield upregulated bio-active compounds comprising unopposed B12, wherein the spirulina product has more bioavailable B12 than pseudo-B12.

One aspect of the present invention provides pharmaceutical compositions, nutritional supplements and/or food products comprising any of disclosed spirulina biomass, products, extracts, water-soluble and/or water-insoluble components thereof, and/or combinations thereof having more bioavailable B12 than pseudo-B12.

One aspect of the present invention provides a method of preparing a food product with enhanced contents of bioavailable B12 from a spirulina product, the method comprising: cultivating Arthrospira spp. cyanobacteria under artificial conditions that include intense illumination and mixing—to yield upregulated bio-active compounds comprising unopposed B12, preparing a spirulina product from the cultivated Arthrospira spp. cyanobacteria, the spirulina product having more bioavailable B12 than pseudo-B12, and preparing the food product from the spirulina product.

These, additional, and/or other aspects and/or advantages of the present invention are set forth in the detailed description which follows, possibly inferable from the detailed description, and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

The patent or application file contains at least one drawing executed in color, e.g., FIGS. 4A-4G, illustrating the colors of the prepared products. Copies of this patent or patent application publication with color drawing(s) will be provided by the Office upon request and payment of the necessary fee.

In the accompanying drawings:

FIGS. 1A-1F provide chromatograms with experimental results that indicate the difference between pseudo-B12 and active methylcobalamin (MeB12) in the disclosed and prior art extracts.

FIG. 2 is a high-level schematic flowchart illustrating cultivation, extraction and treatment methods, according to some embodiments of the invention.

FIGS. 3A-3D are high-level schematic illustrations of cultivation systems, according to some embodiments of the invention.

FIGS. 4A-4G illustrate non-limiting examples of using disclosed spirulina biomass and/or dry/wet extracts in various foods and drinks, according to some embodiments of the invention.

These, additional, and/or other aspects and/or advantages of the present invention are set forth in the detailed description which follows; possibly inferable from the detailed description; and/or learnable by practice of the present invention.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well-known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Some embodiments of the present invention provide efficient and economical methods and mechanisms for preparing spirulina extracts and powders with a high level of unopposed, active vitamin B12 (bioavailable B12 minus pseudo B12), and thereby provide improvements to the technological field of nutritional supplements and improving bioavailable B12 content in food and other products. It is noted that, while spirulina extracts are typically prepared from cyanobacteria such as Arthrospira spp., the term algae (as in blue-green algae) is also commonly used to denote the cyanobacteria due to their photosynthetic capabilities and chlorophyll content. While in some of the results disclosed herein bioavailable, unopposed B12 was measured as methylcobalamin (MeB12), disclosed extracts also include other forms of bioavailable, unopposed B12, such as 5-deoxyadenosylcobalamin, which is another form of metabolically active vitamin B12 (contrasted with pseudo-B12 which are forms that are not metabolically available).

Unexpectedly, the artificial growing conditions within the bioreactor, including intense illumination and mixing, resulted in modifying the metabolic pathways of the cyanobacteria with respect to B12—from producing mainly pseudo B12 that is not bioavailable, into from producing mainly unopposed B12 that is bioavailable—thus turning the corresponding spirulina products from being anti-nutrients with respect to B12 (as prior art spirulina products include mainly pseudo-B12 which opposed absorption of B12)—into being nutritious with respect to B12—providing bio available vitamin B12.

Spirulina products, biomass, extracts, various food products, nutritional supplements, and methods of preparing and using the spirulina products are provided—in which bioavailable B12 outweighs pseudo B12. The spirulina products are prepared from Arthrospira spp. biomass that is cultivated under controlled, ultra-high-density conditions with strong UV illumination and strong continuous mixing. Non-limiting examples for the photosynthetically controlled conditions under which the Arthrospira spp. was cultivated include temperatures under 31±2° C., pH of 10.8±0.2 and irradiance of between 700-1,500 mol/m²s, subranges thereof, or possibly even higher irradiance. Specifically, as disclosed below, cultivation of the Arthrospira spp. in an ultra-high-density culture having a density between 3 g/l and 10 g/l and under ultraviolet radiation intensity of between 70-150 mol/m's was found to yield the disclosed characteristics for use in the disclosed spirulina products.

The spirulina products may be made of the algal biomass and/or wet/dry extracts thereof, and are characterized by high levels of unopposed B12 (e.g., methylcobalamin, 5-deoxyadenosylcobalamin or other metabolically active forms of B12), providing more bioavailable B12 than pseudo-B12. Spirulina products may have more bioavailable B12 than pseudo-B12 (e.g., at least two or three times more bioavailable B12 than pseudo-B12)—providing a large portion of the required daily B12 intake in a broad range of food products (e.g., 15% or more of the daily value, DV).

The spirulina products may be prepared from any of the water-based algal extract without further processing, dried extracts, spirulina biomass, dried biomass, spirulina paste, spirulina powder and/or spirulina liquid. Water-based extracts may be prepared by water extraction of Arthrospira spp. biomass that is cultivated under controlled, ultra-high-density conditions with strong UV illumination and strong continuous mixing, and are characterized by high levels of unopposed B12 (e.g., methylcobalamin, 5-deoxyadenosylcobalamin or other metabolically active forms of B12), as contrasted with prior art B12 supplements that include large amounts of pseudo-B12. Water-based spirulina extracts may be produced by water-extraction and cycles of freezing and thawing applied to the cultivated Arthrospira spp.

The spirulina products may correspondingly be used as food products and/or nutritional supplements or food product fortifications—as sources for bioavailable B12. Various food products may include disclosed spirulina products, biomass, extracts, spirulina powders, pastes and liquids, water-soluble and/or water-insoluble components thereof, and/or combinations thereof. Examples for food products may include meat analogues, baked goods, frozen food products, functional bars, gummies, various drinks (e.g., shakes, smoothies) and drink additives, effervescent tablets, multilayered capsules, e.g., for using in various drinks and beverages, as well as combinations thereof. Nutritional supplements and additives may comprise the disclosed products and/or fractions thereof and may be used as standalone supplements or be added to various food products, e.g., as disclosed herein. Moreover, disclosed spirulina products may be used in food products to directly decarbonize them in the sense suggested by Tzachor et al. 2022, in addition to providing bioavailable B12 and additional nutritional elements. For example, bakeries may decarbonize their manufacturing line by adding disclosed spirulina products to the bread and baked goods to simultaneously enhance their nutritional value by providing bioavailable B12 and reduce their carbon footprint, utilizing the negative carbon footprint of the disclosed spirulina products to balance carbon emissions from the bakery. Various types of food products may be fortified with disclosed spirulina products of any suitable form, to enhance their nutritional content, especially of bioavailable B12, and possibly simultaneously to reduce their carbon footprint.

Table 1 provides the results of liquid chromatography with tandem mass spectrometry (LC-MS/MS) analysis of two types of spirulina extracts that were grown and extracted as disclosed below, with a prior art spirulina extract from algae grown in open ponds, obtained as a commercial product. Measurement methods were as described, e.g., in Sela et al. 2020, Wolffia globosa-Mankai Plant-based protein contains bioactive vitamin b12 and is well absorbed in humans, Nutrients 12(10), 3067. Disclosed spirulina extracts were dried to form spirulina powders, which comprise a water-soluble fraction (typically blue in color) and a water insoluble fraction (typically green in color), which are present at a 1:1 ratio in the disclosed spirulina extracts and powders. The data compare, in ng/mg powder, the content of pseudo-B12 molecules, which are physiologically inactive and even detrimental in blocking the B12 receptors, and the content of active MeB12, and illustrate that disclosed extracts and powders have a much higher content of bioavailable MeB12 compared to pseudo-B12, and a high content of bioavailable MeB12 compared to prior art extracts and powders, which include only pseudo-B12.

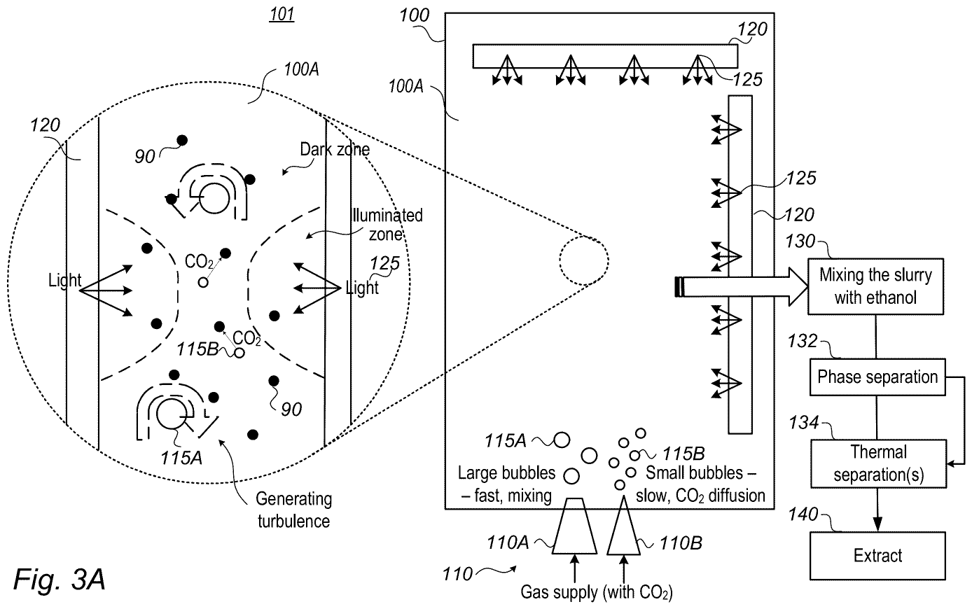

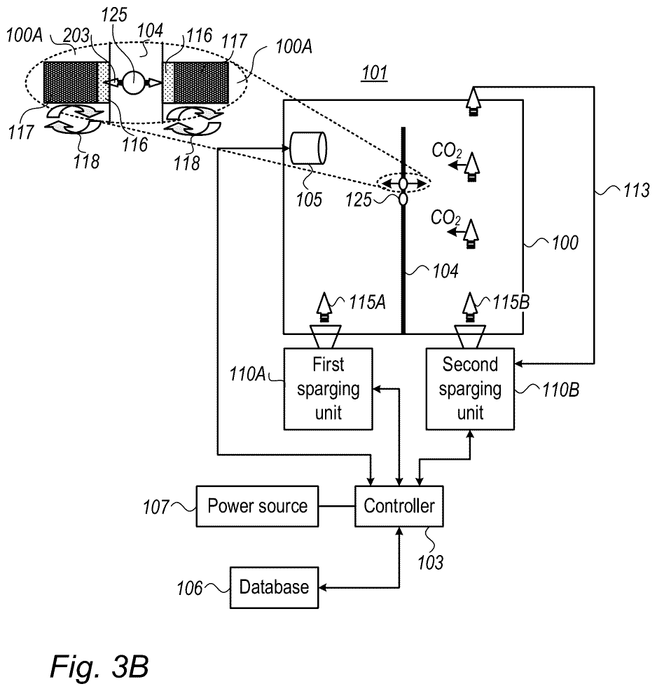

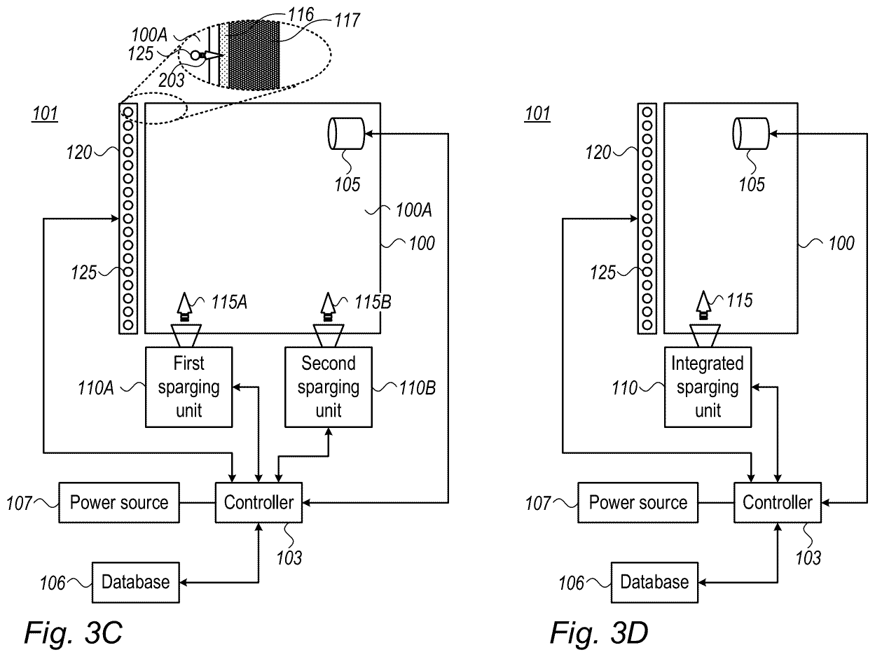

Moreover, Table 1 provides data for two types of illumination regimes, within the 12 h illumination period—(i) a 100-500 ms (milliseconds) exposure period per cell, resulting from the strong mixing and agitation of the cells that brings them in proximity to the illumination sources for short periods (see the dark and illuminated zones illustrated schematically in FIG. 3A), and (ii) a 10-99 ms exposure period per cell, resulting from even stronger mixing and agitation achieved through higher bubbling rates, e.g., of larger bubbles 115A that improves mixing (see the schematic illustration in FIG. 3A).

As is evident from Table 1, in both illumination regimes, the amount of bioavailable B12 is overwhelmingly larger than the amount of pseudo-B12, reaching factors of ca. 3-9 in various portions of the extract in the 100-500 ms exposure period per cell illumination regime, and of ca. 12-20 in various portions of the extract in the 10-99 ms exposure period per cell. The dominance of bioavailable B12 in disclosed extracts and products is in stark contrast to the undetectable amounts of bioavailable B12 in prior art extracts and products.

It is further pointed out that modifying the illumination regime to more intense agitations and shorter cell exposure periods also increased the amount of bioavailable B12 per serving by factors of ca. 2.5-6 in various portions of the extract. These data further emphasizes the surprising importance of the growing conditions in determining the internal metabolic pathways of the algae, which result in these data in unprecedented dominance of bioavailable B12 in spirula extract, in total contrast to prior art spirulina extracts grown under different conditions.

It is noted that, in disclosed extracts, the concentration per ml extract is about five times the concentration per mg powder (the powders comprise dried corresponding extracts). Additional comparison is made in total MeB12 per 100 g serving, including comparison to beef. The data clearly show the approximately one-thousand-fold higher content of bioavailable MeB12 in disclosed products, making them useful as additives and supplements in various uses (e.g., as nutritional supplements and/or as additives to meat analogues due to their high levels of bioavailable MeB12, the lack of which being considered a disadvantage of prior art meat analogues).

TABLE 1
Bioavailable MeB12 and pseudo-B12 in disclosed spirulina
products compared to existing products.

| | | | | | | Unopposed, |
|---|---|---|---|---|---|---|
| | | | Bio- | | | active |
| | Illumination | Pseudo- | available | | | MeB12 per |
| | conditions | B12 | MeB12 | Difference | | serving |
| Sample | (12 h in total) | (ng/mg) | (ng/mg) | (ng/mg) | Ratio | (mcg/100 g) |
| Spirulina | 100-500 ms | 4.18 | 24.21 | 20.03 | 5.79 | 2003.5 |
| biomass (50:50) | exposure | | | | | |
| Water soluble | period per | 4.86 | 14.92 | 10.06 | 3.07 | 1006.0 |
| fraction (blue) | cell | | | | | |
| Water insoluble | | 3.50 | 33.50 | 30.01 | 8.58 | 3001.0 |
| fraction (green) | | | | | | |
| Spirulina | 10-99 ms | 4.9 | 78.0 | 73.1 | 16.1 | 7,310.0 |
| bimass | exposure | | | | | |
| (50:50) | period per | | | | | |
| | cell | | | | | |
| Water soluble | | 5.3 | 66.7 | 61.4 | 12.6 | 6,140.0 |
| fraction (blue) | | | | | | |
| Water soluble | | 4.4 | 89.2 | 84.8 | 20.3 | 8,480.0 |
| fraction (green) | | | | | | |
| Prior art | >100 ms | 0.81 | Not detectable | | | 3.2* |
| product | | | | | | |

Comparable to beef - USDA data: 2.6 MeB12 mcg/100 g for a cooked and broiled ground beef patty (85% lean meat/15% fat)

It is noted that the bioavailable MeB12, being in higher quantity and/or concentration than pseudo-B312, is present in the water soluble portion as well as in the water insoluble portion, and may be used with or without disruption of the cell walls. Accordingly, various embodiments comprise food products and/or nutritional supplements comprising the disclosed spirulina biomass, extracts (dry or wet), products and/or fractionated compounds thereof, e.g., in biomass, extract, paste, liquid or powder form and having more bioavailable MeB12 than pseudo-B312 in the powders or other dry products, or at least 50 ng/ml unopposed MeB12 in the biomass, extract or other liquid products. Various embodiments of food products comprise, e.g., meat analogues, baked goods, frozen food products, ice creams, functional bars, gummies, drinks, shakes, drink additives, effervescent tablet, etc. comprising the disclosed spirulina biomass, extracts, pastes, liquids, powders and/or fractionated compounds thereof.

In various embodiments, food products and/or nutritional supplements comprising the disclosed spirulina biomass, extracts (dry or wet), products and/or fractionated compounds thereof, e.g., in biomass, extract, paste, liquid or powder form, may have unopposed B12 concentrations (e.g., of methylcobalamin, 5-deoxyadenosylcobalamin or other metabolically active forms of B12) of at least 0.7 μg/100 g, at least 2 μg/100 g, at least 10 μg/100 g, at least 10 μg/100 g, at least 100 μg/100 g, at least 1000 μg/100 g, at least 2000 μg/100 g or at least 3000 μg/100 g of unopposed B12 (and more bioavailable B12 than pseudo-B12).

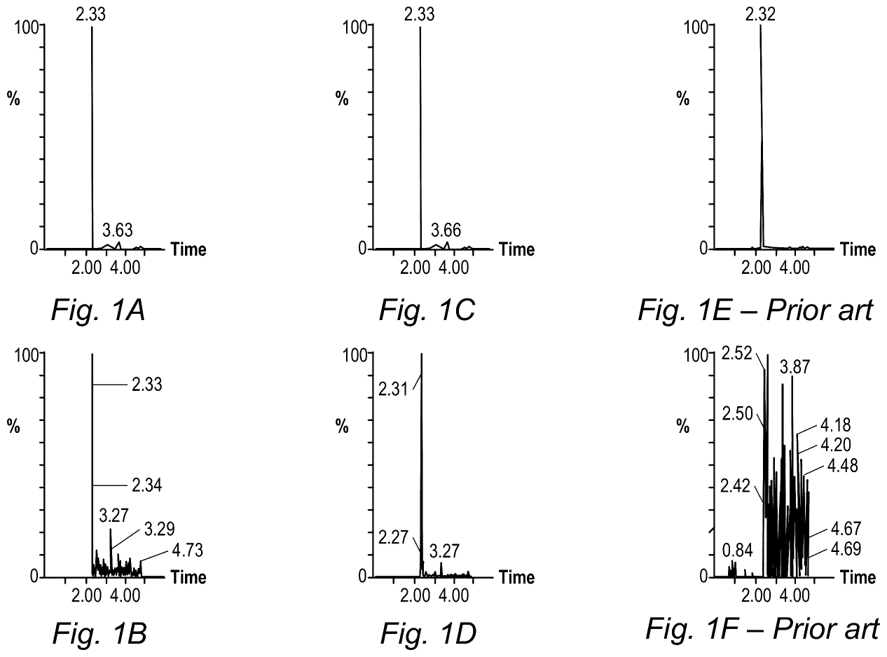

FIGS. 1A-1F provide chromatograms with experimental results that indicate the difference between pseudo-B12 and bioavailable MeB12 in the disclosed and prior art extracts. Specifically, FIGS. 1A and 1B provide chromatograms for bioavailable MeB12 and pseudo-B12, respectively, in the water soluble (blue) fraction of the disclosed extracts; FIGS. 1C and 1D provide chromatograms for bioavailable MeB12 and pseudo-B12, respectively, in the water insoluble (green) fraction of the disclosed extracts—both pairs of graphs indicating the purity of unopposed MeB12 in the disclosed extracts, while FIGS. 1E and 1F provide chromatograms for bioavailable MeB12 and pseudo-B12, respectively, in prior art extracts indicating the large extent of additional MeB12-like compounds termed pseudo-B12 (see the multiple peaks in FIG. 1F), which are not bioavailable and even oppose the bioavailability of MeB12. As is evident in the data, disclosed extracts and fractions thereof yield MeB12 which is much more pure than prior art extracts/powders and commercial products from algae grown in open ponds, under different conditions than presently disclosed. The inventors suggest that the high purity results from the growing and extraction conditions of the disclosed extracts, and may be responsible for the high bioavailability of MeB12 in the disclosed extracts. For example, the inventors suggest that the controlled light conditions (spectral composition, intensity, photo-modulation) may activate different metabolic pathways in spirulina/Arthrospira cyanobacteria that may enable or enhance the synthesis of unopposed methylcobalamin.

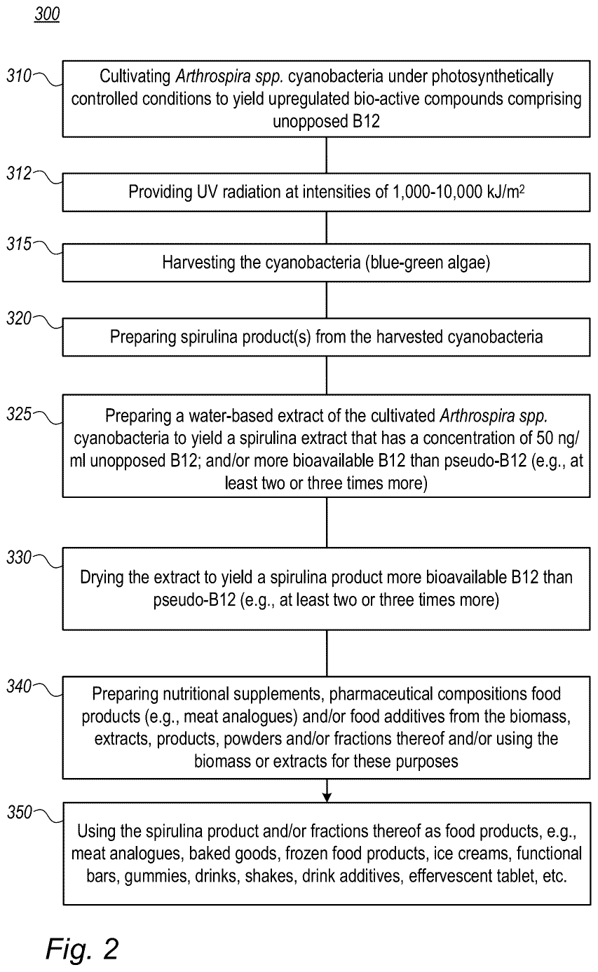

FIG. 2 is a high-level schematic flowchart illustrating cultivation, extraction and treatment methods 300, according to some embodiments of the invention. The method stages may be carried out with respect to cultivation system 101 described herein, which may optionally be configured to implement method 300. Method 300 may be at least partially implemented by at least one computer processor, e.g., in controller 103 comprising corresponding processing unit(s). Certain embodiments comprise computer program products comprising a computer readable storage medium having computer readable program embodied therewith and configured to carry out the relevant stages of method 300. Method 300 may comprise the disclosed stages, irrespective of their order.

As illustrated schematically in FIG. 2, method 300 of preparing a spirulina product may comprise cultivating Arthrospira spp. cyanobacteria under photosynthetically controlled conditions to yield upregulated bio-active compounds comprising unopposed methylcobalamin B12 (MeB12) (stage 310), e.g., in cultivation system 101. Illumination may be provided by at least one of light sources 125, configured to emit UV light at both UVA and UVB spectra. In some embodiments, the ratio between the emitted intensities of UVA/UVB radiation may be in a range of 10-15, for example, 10 UBA/UVB. In certain embodiments, method 300 may be used to grow cyanobacteria, e.g., Arthrospira spp., from which spirulina products are produced. Method 300 may further comprise providing the UV radiation at intensities of 1,000-10,000 kJ/m² (stage 312), for example, 5000 kJ/m² or at any other intermediate value. For example, controller 103 may be configured to control the provision of the UV radiation using on/off radiation pulses. In some embodiments, each pulse may last 0.0099 sec, and between 1-100 of such pulses may be provided per second, for example, 10 times per second. It is noted that approximately 0.01 sec of 1,000 kJ/m² illumination yields about ten times the intensity of solar UV radiation, which changes the chemical composition of the algae and/or cyanobacteria to yield products, extracts and compositions disclosed herein.

Method 300 further comprises harvesting the cyanobacteria (blue-green algae) (stage 315), e.g., implementing continuous harvesting and matching the harvest rate to the growth rate. Method 300 further comprises preparing spirulina product(s) from the harvested cyanobacteria (stage 320), e.g., from the algal biomass and/or from wet or dry extracts thereof. In various embodiments, the spirulina product may be prepared from at least one of: the water-based extract without further processing, dried extracts, spirulina biomass, dried biomass, spirulina paste, spirulina powder and/or spirulina liquid.

For example, method 300 may comprise preparing a water-based extract of the cultivated Arthrospira spp. cyanobacteria to yield a spirulina extract that has a concentration of at least 50 ng/ml unopposed B12 and/or more bioavailable B12 than pseudo-B12 (stage 325), e.g., at least two or three times more. Unopposed, or bioavailable B12 refer to the metabolically active forms of vitamin B12 such as methylcobalamin and 5-deoxyadenosylcobalamin, in contrast to pseudo-B12 which represents metabolically inactive forms.

The extraction may be carried out by applying one or more freeze-thaw cycles to break the cell walls and enhance the extractability of the suspension. For example, the harvested biomass may be rapidly frozen to −20° C. then thawed at 0 to 4° C. until completely de-frosted. Method 300 may further comprise drying the extract to yield a spirulina powder having more bioavailable B12 than pseudo-B12 (stage 330), e.g., at least two or three times more. Products, biomass, extracts (wet or dry) or derivatives thereof may be prepared as described herein.

Method 300 may further comprise preparing nutritional supplements, pharmaceutical compositions food products (e.g., meat analogues) and/or food additives from the biomass, extracts (wet or dry), powders and/or fractions thereof and/or using the biomass or extracts for these purposes (stage 340) and/or using the spirulina products, biomass, extracts (wet or dry) and/or fractions thereof as food products, e.g., meat analogues, baked goods, frozen food products, ice creams, functional bars, gummies, drinks, shakes, drink additives, effervescent tablet, etc. (stage 350). Using the spirulina products in any of these products may also contribute to reducing their carbon footprint, through the negative carbon footprint of growing and processing spirulina.

In certain embodiments, a wet biomass of spirulina may be suspended in pure water (hot or cold) to obtain a product with 10 weight % dry substance. The insoluble substances may be removed by continuous centrifugation. The supernatant, containing soluble biologically active substances, may be used as botanical extracts, which may comprise water-soluble pigments (e.g., phycocyanin), proteins. nucleic acids, polysaccharides and ash. These compounds may be further fractionated (e.g., by chromatography and ethanol precipitation) in order to enhance the content of bioavailable (e.g., unopposed) B12. While, in some embodiments, the harvested biomass may be used directly, e.g., orally, advantageously using the extract and/or fractionated compounds thereof allows using smaller amounts for daily consumption.

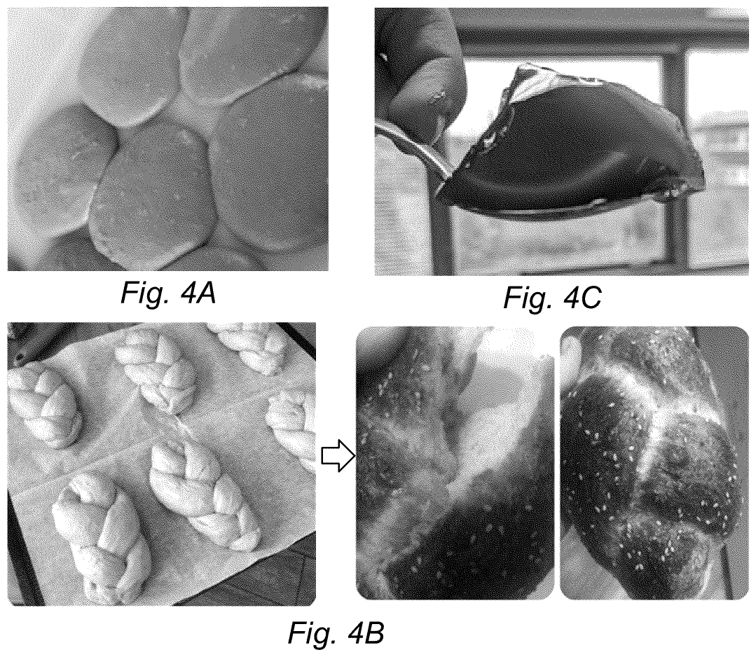

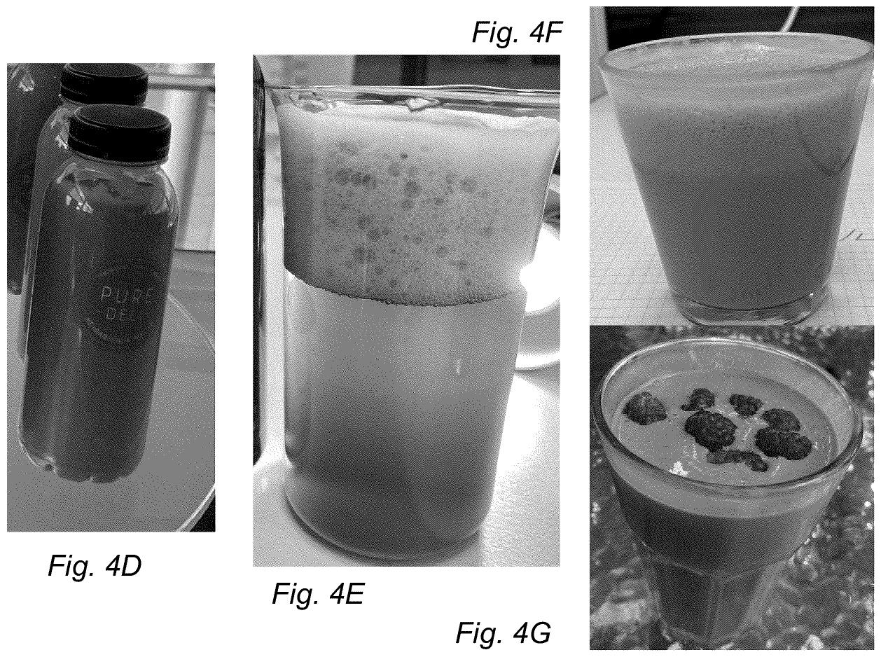

The disclosed products, extracts and/or fractionated compounds thereof may be used as is in nutritional supplements, e.g., in powder form and having more bioavailable B12 (e.g., methylcobalamin or 5-deoxyadenosylcobalamin), than pseudo-B12. It is noted that the bioavailable B12 is present in the water soluble portion as well as in the water insoluble portion, and may be used with or without disruption of the cell walls. Alternatively or complementarily, the disclosed extracts and/or fractionated compounds thereof may be used in pharmaceutical compositions (by themselves or as additives to other pharmaceutical compositions), e.g., with pharmaceutical acceptable carrier(s). Uses of the disclosed biomass, extracts and/or fractionated compounds thereof include uses as nutritional supplement(s) and/or as pharmaceutical compositions and/or as food products, e.g., as additives in meat analogues, shakes and/or ice creams, as well as baked goods (see an example in FIG. 4), frozen food products, functional bars, gummies, various drinks and drink additives or combinations thereof. The spirulina products may be used in form of biomass, extract (wet or dry), paste, liquid, powder or any other form. In various embodiments, disclosed food products may include any of the spirulina extracts, spirulina powders, water-soluble and/or water-insoluble components thereof, and/or combinations thereof. Additional examples for food products include effervescent tablets with spirulina extracts, phycocyanin-enhanced spirulina extracts and multilayered capsules, e.g., for using in various drinks and beverages.

FIGS. 3A-3D are high-level schematic illustrations of cultivation systems 101, according to some embodiments of the invention. Cultivation system 101 is configured to grow algae and/or cyanobacteria in one or more bioreactor(s) 100, at high density and under high illumination intensity. Elements from FIGS. 3A-3D may be combined in any operable combination, and the illustration of certain elements in certain figures and not in others serves merely an explanatory purpose and is non-limiting. It is noted that any disclosed value may be modified by +10% of the value.

FIG. 3A is a high-level schematic illustration of a cultivation systems 101 with bioreactor(s) 100 and extraction equipment for extracting botanical extracts 140, according to some embodiments of the invention. Bioreactor 101 comprises one or more tanks filled with a growth medium 100A (e.g., water, optionally with additives) and Nannochloropsis algae 90 (e.g., N. oculata, N. australis, N. gaditana, N. granulate, N. limnetica, N. oceanica, N. salina, Nitzschia paleacea, Phaeodactylum tricornutum, Pavlova lutheri, Rebecca salina, any strains or combinations thereof, or equivalent algae), and further comprising (i) an illumination system 120 that comprises multiple intense light sources 125, e.g., light emitting diodes (LEDs, e.g., at between 400-700 nm, within sub-ranges thereof, e.g., 400-500 nm, 500-600 nm, 600-700 nm, and/or at specific wavelengths e.g., 650 nm) that may be arranged in one or more horizontal and/or vertical panels (illustrated schematically and only in part), and (ii) a bubbling system 110 associated with a gas supply (e.g., air or nitrogen enriched with CO₂, e.g., including a CO₂ concentration of 30% or higher), which comprises multiple spargers of at least two types—sparger(s) 110A with large nozzles (e.g., >1 mm in diameter, possibly within a range of 1-5 mm or within subranges thereof) for generating large bubbles 115A that move fast (e.g., 100 min⁻¹, indicating the rate of cumulative bubble volume to container volume, possibly within a range of ±30%, of 50-150 min⁻¹ or within subranges thereof) through the algae culture and mix it, and sparger(s) 110B with small nozzles (e.g., <1 mm in diameter, possibly within a range of 0.1-1 mm or within subranges thereof) for generating small bubbles 115B that move slowly (e.g., 5 min⁻¹, indicating the rate of cumulative bubble volume to container volume, possibly within a range of ±30%, of 2-30 min⁻¹ or within subranges thereof) through the algae culture and enable CO₂ to diffuse to the algal cells. Possibly more than two types of nozzles may be used to control the mixing of the algae culture and the delivery of CO₂ at a sufficient concentration to the algae. The two or more types of spargers (each with multiple nozzles) may be distributed at one or more locations and may be configured to generate turbulent mixing of the algae in the cultivation container and provide CO₂ to the algae to the extent that maximizes or optimizes their growth and/or their content and/or composition, e.g., maximize the content and/or composition of unopposed B12 (e.g., methylcobalamin or 5-deoxyadenosylcobalamin) and/or enhances the bioavailability of B12.

As illustrated schematically in the enlarged region of FIG. 3A, the very dense algae culture (e.g., having an algal density of at least 5 g/l, possibly within a range of 5-15 g/l or within subranges thereof) and localized intense light sources 125 create illuminated zones and dark zones within bioreactor 100, and the intense agitation of the algae culture by large bubbles 115B continuously mixes the liquid and moves algal cells 90 between the dark and the illuminated zones. In non-limiting examples, light sources 125 may reach an illumination intensity of any of at least 700 micromole·m⁻²s⁻¹, at least 1000 micromole·m⁻²s⁻¹, at least 1200 micromole·m⁻²s⁻¹, or intermediate values each, e.g., using at least 24 LEDs over an area of 4 m² with a light path of about 2.5 cm—defining the reach of the illuminated zones). The intense non-homogenous illumination, dark zone periods, and high level of CO₂ fed to algal cells 90 by small bubbles 115A yield a high growth rate of the algae, and was also found to modify the biosynthetic pathways employed by the algae to form organic compound. For example, at high density conditions, the illuminated zones may extend to a few millimeters (e.g., 1-5 mm) from point sources 125, while the dark zones between the illuminated zones may extend over a few tens of millimeter (e.g., 20-30 mm) between consecutive illuminated zones, so that individual algae cells spend periods in dark zones to assimilate CO₂ using the light energy absorbed in the illuminated zones.

The growing conditions may be monitored to maintain optimal growth, e.g., the temperature may be kept constant (e.g., at any of 15° C., 20° C., 25° C., 27° C., between any of 12-17° C., 15-20° C., 20-25° C., 25-30° C., 20-40° C., 20-50° C. or within subranges thereof) or be allowed to change within a specified range, chemical conditions such as the pH, O₂ and CO₂ content and/or content of various ions or compounds may be kept constant or be allowed to change within a specified range (e.g., between pH values of 6.7 and 7.2, or any subranges thereof). For example, the organic carbon content may be maintained at or around 20 wt %. Alternatively or complementarily, CO₂ concentration may be kept at or below 40%, and/or the pH may be monitored to indicate and regulate the CO₂ concentration. Algae density may likewise be kept constant or be allowed to change within a specified range. The flow rates of small bubbles 115B from sparger(s) 110B with the small nozzles may be adjusted to increase or reduce CO₂ levels (e.g., increased above 5 min⁻¹, e.g., to 7-10 min⁻¹ to increase CO₂ levels, or reduced to 2 min⁻¹ to reduce CO₂ levels). In certain embodiments, the level of various nutrients may be monitored, and additional nutrients may be provided via one or both bubble streams, e.g., phosphorous may be added to the gas supply if a low P level is detected or if growth is inhibited.

Algal slurry may be periodically or continuously removed from bioreactor 101, e.g., to balance the growth of biomass, as indicated schematically in FIG. 3A. Disclosed spirulina products may comprise a botanical extract 140 that may be produced by mixing the algal slurry with ethanol (stage 130) and then extracted from the mixture, e.g., by one or more stages of phase separation (stage 132) and thermal separation (stage 134). Disclosed spirulina products may comprise one or more of the separated phases. Non-limiting examples for parameters of the separation process include a volume ratio of at least 5:1 (or possible within the range 3.5:1 to 7:1 or intermediate subranges) between ethanol and the algal slurry (the mixture may be stirred for between 0.5-4 hours, e.g., for 2 hours), use of centrifugation for the phase separation, possibly under vacuum (e.g., under a pressure between 75-125 mbar) and a temperature range of between 55° C.-65° C. for the thermal separation. In certain embodiments, up to 90% of the algal mass may be removed per day.

In particular, the inventors have surprisingly found that extract 140 yielded by separation stages 132, 134 comprises more bioavailable B12 than pseudo-B12 (e.g., at least two or three times more bioavailable B12 than pseudo-B12). Accordingly, botanical extract 140 may be kept in its relatively pristine form, avoiding high temperatures and chemical modification.

As additionally illustrated schematically in FIGS. 3B-3D, cultivation system 101 comprises at least one first sparging unit 110A having a plurality of nozzles and configured to distribute a first predetermined fluid 115A (e.g., air and/or nitrogen bubbles) into a water-filled algae cultivation container 100 (e.g., a bio-reactor) at a first operating flow rate so as to allow mixing therein (indicated schematically by arrows 118). Cultivation system 101 may further comprise at least one second sparging unit 110B having a plurality of nozzles and configured to distribute a second predetermined fluid 115B (e.g., gas bubbles with CO₂, indicated schematically, and/or dissolved phosphorus for mass transfer) into container 100 at a second operating flow rate. Fluids exiting container 100, such as gas from second predetermined fluid 115B may be recycled 113 to fully utilize remaining CO₂ therein (illustrated schematically).

Cultivation system 101 may further comprise at least one controller 103 in communication with sparging units 110 and configured to control the first operating flow rate and the second operating flow rate provided thereby. Controller 103 may comprise one or more processing units that implement computer code. For example, at least one nozzle of first sparging unit 110A and/or at least one nozzle of second sparging unit 110B may be configured to distribute fluid into cultivation container 100 based on instructions from at least one controller 103. In some embodiments, the first operating flow rate may be based on the second operating flow rate and/or at least one of the operating flow rates may be predetermined. In some embodiments, the first operating flow rate may be adapted to allow turbulent mixing of the algae in cultivation container 100. In some embodiments, the second operating flow rate may be adapted to allow mass transfer and/or assimilation of materials in a liquid in cultivation container 100. Information may flow between controller 103 and sparging units 110, as well as between controller 103 and other elements in the system, as indicated schematically by the arrows.

Second predetermined fluid 115B may include gas bubbles with over 30% CO₂ concentration. The source for the first predetermined fluid(s) and/or for the second predetermined fluid(s) may be external to cultivation system 101, for example geothermal power stations may provide a source of dissolved carbon and/or sulfur for the second predetermined fluid.

The first operating flow rate of at least one nozzle of first sparging unit 110A (e.g., 100 ml/min) may be different from the second operating flow rate of at least one nozzle of second sparging unit 110B (e.g., 5 ml/min). In some embodiments, at least one nozzle of first sparging unit 110A may have a diameter larger than about 1 millimeter. In some embodiments, at least one nozzle of second sparging unit 110B may have a diameter smaller than about 1 millimeter. In some embodiments, nozzles of first sparging unit 110A as well as of second sparging unit 110B may distribute the same fluid (e.g., air), with nozzles of each sparging unit having different diameters. Larger apertures of first sparging unit 110A may be configured to provide first predetermined fluid 115A with large bubbles (e.g., of air and/or nitrogen) to agitate and mix 118 the suspended biomass in container 100, while smaller apertures of second sparging unit 110B may be configured to provide second predetermined fluid 115B with small bubbles (e.g., of or comprising CO₂) to transfer CO₂ from the gas to the liquid to be accessible to the suspended biomass in container 100. Advantageously, the difference in the size of the delivered bubbles may prevent combination of the bubbles of streams 115A, 115B, providing simultaneously mixing 118 by the high throughput of big and fast bubbles in stream 115A and effective CO₂ supply by the small throughput of small and slow bubbles in stream 115B.

Cultivation system 101 may further include a physical barrier 104 configured to separate the first fluid distributed by first sparging unit 110A and the second fluid distributed by second sparging unit 110B within cultivation container 100. In some embodiments, at least one nozzle of first sparging unit 110A and/or of second sparging unit 110B may be embedded into physical barrier 104 (not shown). In some embodiments, physical barrier 104 may be adapted to allow flow from one side of barrier 104 (having a first fluid distribution) to the other side of barrier 104 (having a second fluid distribution) at predefined (e.g., upper and lower) locations of cultivation container 100, in order to create a controlled flow within the container 100.

Cultivation container 100 with physical barrier 104 may include at least one light source 125 embedded into the physical barrier 104 such that container 100 may be illuminated (illumination denoted schematically by arrows 203) from within by from at least one light source 125 embedded into the physical barrier 104. Cultivation container 100 may include a plurality of physical barriers 104, each including at least one light source 125, such that a modular system may be created with algae and/or cyanobacteria growing between adjacent physical barriers 104, wherein at least one controller 103 may control illumination for all light sources 125 embedded into the physical barriers 104. In some embodiments, physical barriers 104 may comprise illumination system 120 with multiple intense light sources 125.

In certain embodiments, cultivation system 101 may be configured to reach a very high density of the cultivated biomass with a corresponding small optical depth that yield a relatively thin illuminated zone 116 and a much thicker dark zone 117 in container 100, with the biomass being continuously agitated 118 (e.g., by strong bubbling of fluids 115A and optionally 115B) so that individual cells of algae and/or cyanobacteria have but a brief residence time in illuminated zone 116 before returning to dark zone 117. In non-limiting examples, the thickness of illuminated zone 116 may be configured to be between 0.1 cm and 1.5 cm, depending on the density of the suspension and the illumination density, and may be controlled by controller 103 and adjusted according to specified requirements. Accordingly, illumination 203 (and particularly UV components thereof) may be set at very high levels as the brief residence time prevents illumination damage to the individual cells.

Cultivation system 101 may further include at least one sensor 105 (e.g., a temperature sensor) coupled to controller 103 and configured to detect at least one feature within cultivation container 100. For example, at least one sensor 105 may be configured to detect any of the pH levels, the temperature, and the pressure conditions within cultivation container 100 and/or sections thereof. In some embodiments, at least one sensor 105 may also be configured to detect parameters external to cultivation container 100, for example measuring mass flow of the gas emissions from cultivation container 100 to determine an amount of substance that was absorbed in the algae cells by subtracting the emitted amount from the amount inserted into the container (e.g., by second sparging unit 102).

Cultivation system 101 may further include at least one database 106 (and/or memory unit) configured to store algorithms for operation of controller 103, for instance database of operating rates for each nozzle and/or each sparging unit. In some embodiments, cultivation system 101 may further include a power source 107 coupled to controller 103 and configured to provide electrical power to cultivation system 101. Power source 107 may be configured to power at least one first sparging unit 110A and at least one second sparging unit 110B, e.g., to operate at different rates.

Data gathered by at least one sensor 105 may be analyzed by controller (or processor) 103 to detect if an attribute exceeds a predetermined threshold, for instance threshold for pH level and/or temperature and/or CO₂ concentration within the container 100. In case conditions within cultivation container 100 (e.g., as detected by sensor 105) exceed at least one threshold, then controller 103 may operate at least one nozzle of first sparging unit 110A and/or at least one nozzle of second sparging unit 110B at a different flow rate. For example, detecting CO₂ concentration within the container 100 exceeds 40% (or detecting low pH levels) may cause at least one nozzle of second sparging unit 110B to lower flow rate of second sparging unit 110B to about 2 millimeters/minute. In some embodiments, at least one nozzle of second sparging unit 110B may operate only upon receiving a signal from sensor 105 that an attribute exceeds a predetermined threshold, and not operated in a constant rate.

At least one nozzle of first sparging unit 110A may be configured to operate only upon receiving a signal from sensor 105 that an attribute exceeds a predetermined threshold, for example increasing mixing flow 118 as the density of algal population increases. At least one nozzle of first sparging unit 101 and/or at least one nozzle of second sparging unit 110B may operate in a constant rate, continuously, or possibly intermittently. At least one nozzle of first sparging unit 110A and/or at least one nozzle of second sparging unit 110B may operate in a non-constant rate continuously, or possibly intermittently.

Cultivation container 100 may comprise a bubble column configuration with at least one first sparging unit 110A and at least one second sparging unit 110B positioned on the same surface of the bubble column container. Cultivation container 100 may have an airlift configuration with at least one second sparging unit 110B positioned at a bottom portion of a downcomer that may be distal to sensor 105, such that residence time of bubbles from at least one second sparging unit 110B may be increased.

Cultivation system 101 may be configured to enable maintaining at least 20% organic carbon within container 100, calculated in addition to carbon provided as CO₂ bubbles. In some embodiments, at least portion(s) of the algae within container 100 may comprise any photosynthetic microorganism such as algae and/or cyanobacteria used to prepare spirulina preparations, including, e.g., Arthrospira platensis, A. fusiformis and/or A. maxima.

In some embodiments, optimized controlled provision of the harmful UV radiation may allow increasing the amounts and/or bioavailability of B12 (e.g., methylcobalamin and/or 5-deoxyadenosylcobalamin), in the cultivated Arthrospira spp. and on the extracted spirulina while avoiding damaging the growing algae or the growing rate. In some embodiments, the on/off nature of the radiation provision may allow controlling the amount of harmful radiation provided. Moreover, the constant mixing and/or bubbling 118 of the suspension in container (by sparging units 110) ensures only brief exposures of any individual algal or cyanobacterial cells to the intense radiation, preventing photoinhibition and damage to the cells. Controller 103 may be respectively configured to control the extent of turbulence provided by sparging units 110 to avoid radiation damage to the cells. For example, method 300 and cultivation system 101 may be configured to achieve the required UV photo-modulation in a thin-film cultivation system, by turning the UV light source on and off and/or by creating shade patterns that yield intermittent illumination of the algae and/or cyanobacteria. In bubbled cultivation systems 101, the relative velocities of the flows of the culture suspension and of the gas bubbles relative to the UV light source may be controlled to achieve specified patterns of on/off UV exposure cycles.

FIGS. 3C and 3D illustrates schematically embodiments of cultivation systems 101, according to some embodiments of the invention. Cultivation system 101 may comprise at least one illumination unit 120 coupled to controller 103, to illuminate cultivation container 100. Illumination unit(s) 120 and controller 103 (or another controller) may be included in a bioreactor illumination system for growing algae and/or cyanobacteria. The distance between cultivation container 100 and illumination unit(s) 120 may be modified to control the illumination received by cultivation container 100. For example, bringing illumination unit(s) 120 closer to cultivation container 100 increases illumination of the culture therein. The distance between cultivation container 100 and illumination unit(s) 120 may be controlled by controller 103, for example, included in the illumination system. In addition to, or instead of, changing the distance of illumination unit 120 from cultivation container 100, the illumination intensity of light sources 125 in illumination unit 120 may be controlled. Illumination unit 120 may include at least one light source 125 (e.g., LED), and each light source 125 may be controlled separately by controller 103. In some embodiments, one or more light source(s) 125 may be controlled to illuminate with a different intensity than another light source(s) 125. All light sources 125 may be controlled to change the illumination intensity, either manually or according to preset timing and/or sensed conditions in cultivation container 100. At least some of light sources 125 may be configured to emit light in the UV spectrum, for example, in both the UVA and the UVB range. The ratio between the emitted radiation in UVA and UVB (UVA/UVB ratio) may be between 10 and 15, e.g., 10, 12, 14, 15 UBA/UVB or have intermediate values. The UV radiation may be provided at 1,000-10,000 kJ/m², for example, 2000 kJ/m², 5000 kJ/m², 7000 kJ/m², 9000 kJ/m² or any other intermediate value. Controller 103 may be configured to control the provision of the UV radiation using radiation pulses, e.g., each pulse may last less than 0.01 sec, e.g., 0.008, 0.009, 0.0095 or 0.0099 seconds, or any intermediate value, and between 1-100 of these pulses may be provided per second, for example, 10 times per second. It is noted that approximately 0.01 sec of 1,000 kJ/m² illumination yields about ten times the intensity of solar UV radiation, which changes the chemical composition of the algae and/or cyanobacteria to yield extract compositions disclosed below. Some light sources 125 may be configured to emit light at the visible spectrum (e.g., wavelength of 400-700 nm).

The amount of light delivered to cultivation container 100 may be defined as an average of light flux delivered to the surface of cultivation container 100. Cultivation system 101 may be used to support ultra-high-density cultures (e.g., having a biomass density of 1, 5 or up to 10 g/l, or intermediate values), with illumination unit(s) 120 configured to have a light distribution of light source(s) 125 that provides an average light flux per algae/cyanobacteria cell that is comparable to average light flux per cell of low-density cultures achieving a similar level of average illumination per cell. Light intensity within cultivation container 100 may be measured with at least one sensor 105 and adjusted by controller 103. For example, for ultra-high-density cultures a typical thickness of illuminated zone 116 may range, e.g., between 1 mm and 5 mm, while a typical thickness of dark zone 117 may range, e.g., between 20 mm and 30 mm). The distance of illumination unit 120 from the side of container 100 may be adjusted with respect to the prevailing optical thickness (or optical depth, OD) of the biomass suspension to avoid photoinhibition and/or photo-bleaching. For example, at initial cultivation stages, when the culture density is relatively low, illumination unit 120 may be initially kept at a distance from the side of container 100 to biomass growth, while at later cultivation stages, once the OD increases, illumination unit 120 may be brought closer to the side of container 100 to promote biomass growth.

Ultra-high-density cultures be continuously and/or intermittently mixed and/or agitated (indicated schematically by arrows 118 in FIG. 3B), by mechanical means and/or by strong bubbling of fluids 115A and optionally 115B, to cause the algae and/or cyanobacteria to move between illuminated zone 116 and dark zone 117 and to prevent damage to the cells, yielding illumination cycles for the algae/cyanobacteria (between illuminated zone 116 and dark zone 117) due to the short light passage. Ultra-high-density cultures may be illuminated with various wavelengths, since in such biomass densities the wavelength may have nearly no effect on the growth due to the short light passage. This is in contrast to the common practice, according to which algae are illumination with specific wavelengths (e.g., with blue light) for normal growth since algae should respond to light differently. However, the inventors have found out experimentally that illumination with any wavelength may be used for ultra-high-density cultures.

The light penetration into cultivation container 100 may correspond to at least one of the light intensity, the light wavelength, the specific algal strain, and/or the algal culture density. It should be noted that the light penetration into cultivation container 110 may determine the volumetric ratio between illuminated zones 116 and dark zones 117 within cultivation container 110, and thus may affect the light intensity provided by illumination units 120, the gas flow rate through first sparging unit 110A, the gas flow rate through second sparging unit 110B, etc., which may be adjusted and optimized respectively.

In some embodiments, cultivation container 100 may be illuminated by illumination unit(s) 201 to provide a daily amount of over 90% of maximal algae growth within the cultivation container 100. In some embodiments, illumination unit(s) 201 may be configured to illuminate the suspension in container 100 in a non-homogenous manner, e.g., using few high intensity light sources 120 spaced apart, because the illumination of the cells is local and temporally controlled (through agitation 118). The inventors have found out that high intensity intermittent illumination actually enhances growth of algae and/or cyanobacteria, over common practice configurations with homogenous distribution of low intensity light sources.

For example, the illumination photon flux density of at least one light source 120 may be 1200 μmole/m²s. In various embodiments, the photon flux density may range between 1000-1500 μmole/m²s or have intermediate values. In some embodiments, illumination unit(s) 120 may include at least four light sources 125 per m². As an illustrative non-limiting example, illumination unit 120 having a surface area of about 6 m², a light path (thickness of illuminated zone 116) of about 4 cm may include 24 LED light sources 125, each having light flux of 1200 μmole/m²s. In some embodiments, at least a portion of the cyanobacteria within container 100 comprises Arthrospira spp. used to prepare spirulina extracts.

Controller 103 may be configured to control the illumination wavelength of the at least one light source 125, for instance with a dedicated illumination module adapted to modify the wavelength of the emitted illumination. In some embodiments, a constant temperature of 27° C. may be maintained within the container 100. In some embodiments, controller 103 may be configured to control at least one light source 125 to illuminate with a wavelength of 650 nanometers. It should be noted that, according to common practice, algae are illuminated with a particular wavelength (e.g., with blue light) for optimal growth, but experiments conducted by the inventors have shown that illumination with other wavelengths (e.g., with red light) may be used for enhanced growth.

As illustrated schematically in FIG. 3D, cultivation system 101 may be configured to operate with an integrated sparging unit 110, which may comprise nozzles of two or more types, integrating sparging units 110A, 110B. Integrated sparging unit 110 may be configured to distribute a predetermined fluid 115 into cultivation container 100. Predetermined fluid 115 may comprise one or both of predetermined fluids 115A, 115B, or various mixtures of parts thereof. For example, integrated sparging unit 110 may include at least one nozzle to distribute first predetermined fluid 115A and at least one nozzle (e.g., having a different diameter) to distribute second predetermined fluid 115B, which in combination are denoted schematically in a non-limiting manner as predetermined fluid 115. Sparging unit 110 may be configured to generate turbulent mixing of the algae and/or cyanobacteria in cultivation container 100, as well as provide CO₂ for assimilation thereby, e.g., as disclosed with reference to FIGS. 3B and 3C. In certain embodiments, cultivation systems 101 may comprise combinations of dedicated sparging units 110A, 110B and integrated sparging unit(s) 110.

The inventors have found out that disclosed spirulina extracts, extracted from algae grown under the conditions disclosed above, contain unopposed, metabolically active B12 (e.g., methylcobalamin and/or 5-deoxyadenosylcobalamin), for example, more bioavailable B12 than pseudo-B12. The extract, biomass, or portions thereof may be used as additives to various food products, providing a significant portion of the daily value of B12 through unopposed methylcobalamin B12, without adding much mass to the products, as the concentration of unopposed B12 is very high in the spirulina product (see, e.g., in Table 1, thousands of times higher than B12 in meat, for example). Accordingly, adding a small amount of disclosed spirulina products, while supplying a significant amount of B12, would not alter the other nutritional or organoleptic properties of the food products.

A long-term study has shown that the content of bioavailable (e.g., unopposed) MeB12 under the disclosed growing conditions was consistent and provided a reliable source for bioavailable MeB12. Table 2 provides results for bioavailable MeB12 and pseudo-B12 in disclosed spirulina biomass tracked over nine months. These results were consistent also over six independent repetitions, yielding similar results. Although both pseudo- and active forms of vitamin B₁₂ were observed, the active form largely dominates the composition (>98%). While the composition of traditional, naturally occurring Spirulina is dominated by pseudo-vitamin B₁₂, with a net-negative content of active B₁₂ (see Table 1), the results obtained here illustrate the effects of photonic management on enhancing natural metabolic pathways for achieving active, and net-active B₁₂ in Spirulina.

TABLE 2
Bioavailable MeB12 and pseudo-B12
in disclosed spirulina biomass.

| | Month #3 | | Month #9 | |
|---|---|---|---|---|
| | μg/100 g | ±SD | μg/100 g | ±SD |
| Pseudo-Vitamin B12 | 0.02 | 6% | 0.04 | 6% |
| Active Vitamin B12 | 1.7 | 4% | 1.7 | 5% |
| Net Active Vitamin B12 | 1.7 | 4% | 1.6 | 5% |

Accordingly, unopposed active vitamin B12 content was found to be statistically consistent over nine months of continued cultivation, yielding a concentration of 1.64 μg/100 g, and thus being comparable or slightly superior to beef meat with a vitamin B12 concentration of 0.7-1.5 g/100 g. Moreover, disclosed spirulina products also provide comparable concentrations of macro- and micro-nutrients (e.g., essential amino acids, alpha-linolenic acid (ω-3 fatty acid) and linoleic acid (ω-6 fatty acid), various minerals, including calcium, potassium, magnesium and iron, and vitamins, such as beta-carotene) and therefore provide good meat-replacement products that are comparable in their nutritional values to meat, but do not have deleterious health implications associated with meat consumption, and moreover have a negative carbon footprint. On the other hand, disclosed spirulina products are also superior to traditional spirulina or other plant-based meat replacements in their nutritional content, and especially in their content of bioavailable, unopposed active vitamin B12.

For example, disclosed spirulina products include a high level of unopposed active vitamin B12 (e.g., 1-3 μg/100 g) and are grown with a slightly negative carbon footprint (−0.008 kg CO₂-eq per kg, see Tzachor et al. 2022), which is a unique combination compared both to beef products and to prior art spirulina products. Compared to beef products, disclosed spirulina products comprise a comparable amount of unopposed active vitamin B12, as well as other nutrients such as comparable protein composition and iron (see Tzachor et al. 2022)—but providing a large carbon credit with respect to beef (e.g., 99.4 kg CO₂-eq per kg for beef, minus 0.008 kg CO₂-eq per kg for disclosed spirulina products—yielding 99.408 kg CO₂-eq per kg as carbon credit). Compared to prior art spirulina products grown under natural or artificial light (rather than under the disclosed high intensity growing conditions)—disclosed spirulina products comprise a high level of unopposed active vitamin B12 as explained.

In case parts of disclosed spirulina products are used as additive, e.g., to meat or dairy replacements, or to baked goods to provide unopposed active vitamin B12—only a part of the negative carbon footprint may be allocated to the product. For example, when replacing meat (with a carbon footprint of 99.4 kg CO₂-eq per kg) using disclosed spirulina products with 17 g/kg unopposed active vitamin B12, each g of unopposed active vitamin B12 may be associated with 5.85 kg CO₂-eq per g unopposed B12 (99.4 kg CO₂-eq per kg/17 g/kg unopposed active vitamin B12)—yielding a reduction in the carbon footprint of the resulting product, in addition to providing unopposed active vitamin B12. For bread, assuming typical values of 1-1.5 kg CO₂-eq per kg bread—adding disclosed spirulina products both increases unopposed active vitamin B12 and reduces the carbon footprint of the bread (e.g., by about 0.1 kg CO₂-eq per g unopposed B12).

FIGS. 4A-4G illustrates non-limiting examples of using disclosed spirulina biomass and/or dry/wet extracts in various foods, according to some embodiments of the invention. Examples in FIGS. 4A and 4B illustrate dough that includes disclosed spirulina, baked into cookies and into bread, respectively, enriching baked goods with bioavailable B12. FIG. 4C is an example of spirulina included in jellies, enriching the jellies with bioavailable B12. Examples in FIGS. 4D and 4E illustrate various types of drinks and shakes, which include disclosed spirulina, enriching them with bioavailable B12. Examples in FIGS. 4F and 4G illustrate smoothies enriched with disclosed spirulina, enriching them with bioavailable B12. The versatile examples for using disclosed spirulina in food products provide natural, bioavailable vitamin B12 to support reduction of tiredness and fatigue, especially in persons lacking B12, e.g., due to nutritional limitations.

In the baked products illustrated in FIGS. 4A and 4B, 0.4 gr disclosed dried spirulina powder was added per 1 kg dough (0.04 wt %, the powder included 30.0 mcg/g bioavailable B12), resulting in 0.6 μg B12 per 50 gr dough, which provides 25% of the recommended dietary intake (RDI). The dough obtained a slightly green hue due to the algae extract powder, which, however, disappeared in the baked bread (FIG. 4B). The dough was baked at 200° C. for 20 minutes, losing 21±3% to evaporation, leaving 0.58±0.04 μg B12 per 50 gr bread serving (which is about 25% of RDI), thus providing an excellent source of B12. The organoleptic properties of the bread did not change with respect to color, smell and taste compared to bread prepared without the spirulina powder.

Similarly, disclosed spirulina biomass and/or dry/wet extracts may be used in a wide range of products (e.g., meat analogues, various baked good, frozen food products, various ice cream types, functional bars, gummies, drinks or shakes, drink additives such as effervescent tablets, or combinations thereof. For example, disclosed products may be prepared to provide a significant part of the daily requirements for B12, e.g., 15% or more.

Moreover, the food products with the spirulina products may have a reduced, zero or negative carbon footprint achieved through the included spirulina product—directly decarbonizing the products in the sense suggested by Tzachor et al. 2022. For example, baked goods may comprise (be fortified with) a water-soluble fraction of the spirulina product, a water insoluble fraction of the spirulina product, and/or fractionated compounds thereof and—in addition to providing bioavailable B12 and additional nutritional elements—have a reduced, zero or negative carbon footprint resulting from adding disclosed spirulina products with a strongly negative carbon footprint—as explained above.

It is noted that disclosed spirulina is grown under conditions which are not available in nature, within bioreactors that deliver intense, continuous illumination and high doses of UV radiation, under continuous mixing. The inventors have found out that these conditions induce different metabolic pathways to operate in the cyanobacteria, as described in detail in Tzachor 2024 (Photonic management of Spirulina (Arthrospira platensis) in scalable photobioreactors to achieve biologically active unopposed vitamin B12, Discover Food, Vol. 4, No. 69, pp. 1-14), which is part of the present study and is incorporated herein by reference in its entirety.

Specifically, the modification of the physiology and metabolic pathways in spirulina was found surprisingly to yield an excess of bioavailable (unopposed) B12 relative to pseudo B12, the latter being the dominant biochemical form of B12 in spirulina grown under solar illumination, as taught by van der Oever and Mayer 2022 (Biologically active or just “pseudo”-vitamin B12 as predominant form in algae-based nutritional supplements?, Journal of Food Composition and Analysis, 10, 104464).

Accordingly, in disclosed embodiments, B12 from spirulina is turned from the prior art anti-nutrient, inhibiting the absorption of B12 though its pseudo B12 content—into disclosed nutrient—providing bioavailable, unopposed vitamin B12 to users. Hence, the disclosed highly artificial growing conditions transform spirulina extracts from anti-nutrients to being highly nutritious with respect to B12.

It is further noted that compared to the inventors' previous discovery of anti-inflammatory effects of minute quantities of spirulina extracts from cyanobacteria grown under artificial conditions (International Patent Application Publication No. WO 2021/234700), the present invention is unexpected, as the metabolic pathways leading to B12, and specifically bioavailable B12 are not related to the pathways leading to the anti-inflammatory effects (specifically being upregulated c-phycocyanin, sorbitol and adenosine derivates which decrease levels of macrophage- and monocyte-induced tumor necrosis factor (TNF)-a), and moreover as the metabolic pathways leading to bioavailable B12 were not known at the time of the previous discovery—as evidenced by Watanabe et al. 1999 and van der Oever and Mayer 2022 and validated by Tzachor 2024.

Finally, the current disclosure of bioavailable B12 from spirulina relates to large doses of B12 supplement, rather than the minute amounts (under 10 μg/ml), which were active in contrast to larger amounts that weren't active, as disclosed in WO 2021/234700.

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A spirulina product made of Arthrospira spp., cultivated under artificial conditions that include intense illumination and mixing, to yield upregulated bio-active compounds comprising unopposed B12, wherein the spirulina product has more bioavailable B12 than pseudo-B12.

2. The spirulina product of claim 1, wherein the spirulina product has at least 0.7 μg/100 g, at least 2 μg/100 g, at least 10 μg/100 g, at least 10 μg/100 g, at least 100 μg/100 g, at least 1000 μg/100 g, at least 2000 μg/100 g or at least 3000 μg/100 g of unopposed B12.

3. The spirulina product of claim 1, wherein the spirulina product has at least two or three times more bioavailable B12 than pseudo-B12.

4. The spirulina product of claim 1, wherein the spirulina product comprises at least one of: a water-based extract of the cultivated Arthrospira spp. without further processing, a dried extract of the cultivated Arthrospira spp., spirulina biomass of the cultivated Arthrospira spp., dried biomass of the cultivated Arthrospira spp., spirulina paste made of the cultivated Arthrospira spp., spirulina powder made of the cultivated Arthrospira spp. and/or spirulina liquid made of the cultivated Arthrospira spp.

5. The spirulina product of claim 1, wherein the photosynthetically controlled conditions comprise at least one of:
cultivating the Arthrospira spp. under the conditions of: 31±2° C., at pH of 10.8±0.2 and under irradiance of between 700 and 1500 mol/(m²s), and
cultivating the Arthrospira spp. in a ultra-high-density culture having a density between 3 g/l and 10 g/l and under ultraviolet radiation intensity of between 70-150 mol/m²s.

6. The spirulina product of claim 1, wherein the photosynthetically controlled conditions are configured to provide a 100-500 ms illumination exposure period per cell regime or a 10-99 ms illumination exposure period per cell

7. The spirulina product of claim 1, wherein the product is a water-based extract that is produced by water-extraction and cycles of freezing and thawing applied to the cultivated Arthrospira spp.

8. A pharmaceutical composition comprising fractionated compounds of the spirulina product of claim 1.

9. A nutritional supplement comprising the spirulina product of claim 1, and/or fractionated compounds thereof.

10. The nutritional supplement of claim 9, comprising at least one of a water-soluble fraction of the spirulina product and a water insoluble fraction of the spirulina product.

11. The nutritional supplement of claim 9, further comprising a pharmaceutically acceptable carrier.

12. A food product comprising a spirulina product that has more bioavailable B12 than pseudo-B12, or and/or fractionated compounds thereof.

13. The food product of claim 12, comprising at least one of: a meat analogue, a baked good, a frozen food product, an ice cream, a functional bar, a gummy, a drink, a shake, a drink additive, an effervescent tablet or combinations thereof.

14. The food product of claim 12, comprising a meat analogue that comprises at least one of: a water-soluble fraction of the spirulina product, a water insoluble fraction of the spirulina product, and/or fractionated compounds thereof.

15. The food product of claim 12, comprising a baked good that comprises at least one of: a water-soluble fraction of the spirulina product, a water insoluble fraction of the spirulina product, and/or fractionated compounds thereof.

16. The food product of claim 12, comprising at least 0.7 μg/100 g, at least 2 μg/100 g, at least 10 μg/100 g, at least 10 μg/100 g, at least 100 μg/100 g, at least 1000 μg/100 g, at least 2000 μg/100 g, at least 3000 μg/100 g of unopposed B12.

17. The food product of claim 12, having a reduced, zero or negative carbon footprint achieved through the included spirulina product.

18. A method of preparing a food product with enhanced contents of bioavailable B12 from a spirulina product, the method comprising:
cultivating Arthrospira spp. cyanobacteria under artificial conditions that include intense illumination and mixing—to yield upregulated bio-active compounds comprising unopposed B12,
preparing a spirulina product from the cultivated Arthrospira spp. cyanobacteria, the spirulina product having more bioavailable B12 than pseudo-B12, and
preparing the food product from the spirulina product.

19. The method of claim 18, wherein the cultivation conditions include agitating the cyanobacteria to reach a 100-500 ms exposure period per cell illumination regime or a 10-99 ms exposure period per cell.

20. The method of claim 18, wherein the spirulina product has at least two or three times more bioavailable B12 than pseudo-B12 and the food product prepared therefrom has a reduced carbon footprint.
